SEQUENCE LISTING (10 sequences)

SEQ ID NO: 1 - protein, 8 aa, Lucilia sericata
LLEYLSMR

SEQ ID NO: 2 - protein, 10 aa, Lucilia sericata
SFDDTLDMLK

SEQ ID NO: 3 - protein, 16 aa, Lucilia sericata
SYEYLLLATHFNSYQK

SEQ ID NO: 4 - protein, 15 aa, Lucilia sericata
SLGNPELPTEWLDLR

SEQ ID NO: 5 - protein, 13 aa, Lucilia sericata
ELDASYQYLAMHK

SEQ ID NO: 6 - protein, 13 aa, Lucilia sericata
VFVNSGTSLMDVR

SEQ ID NO: 7 - protein, 10 aa, Lucilia sericata
ANFNVVHESS

SEQ ID NO: 8 - protein, 205 aa, Glossina morsitans
MMKLIVTLCILAVGSQIVHGEMKCSIGNPELPTEWIDLRGECLKAMRDQIQKEIDASYTYLAMGAHFSRDTINRPGFAEHFFKAAKEERQHGAKLIEYLSMRGQLTDDVTDLIMVPTVSKHEWSSGTEALEDALRLETDVTKSIRKLIQTCERKHNYYHLVDWLTGVYLEEQLHGQRDLAGKISTLKKMMDNHGGLGEFLFDKEL

SEQ ID NO: 9 - protein, 222 aa, Glossina morsitans
MKFLIFVALLASSCVLLKAEEVCHNNVVRACSTSTLSGPSICNARYGGISHVEPELQAYINSHLTKSYEYLLLATHFNSYQKNRPGFQKLYQSLSDRSFDDTIDMIKQLTRRGGKADFNTRHESPASVSTQQQRLEVDELHSLAMALDNEKQLTTGAFHVHTQSLHAARDPETAQYIEEKFLGSQAETIRKLSGYANDLAKLMNQPDPSLAIYLFDEYLQKQ

SEQ ID NO: 10 - protein, 224 aa, Drosophila melanogaster
MKLLVAFALIASLGALAQAEEEYCHNSVVTACSSSTFSGNSICNARFAGIEHVEPEVQAYINSQLTKSYEYLLLATHFNSYQKNRPGFQKLYQGLSDRSFDDSIALIKQITKRGGIVDFNTRHESPASVSTQRPTLEVDELHSLALALDNEKQLATGATHIHTRAIHATERDPEMAHYMEEEYLGKQADSVRKLSGYANDLAKLMKVPDPSLSIYLFDEYLQKQ

(19) United States
(12) Patent Application Publication
(10) Pub. No.: US 2009/0304668 A1
(43) Pub. Date: Dec. 10, 2009

(54) LARVAL POLYPEPTIDES HAVING A NUCLEASE ACTIVITY

(75) Inventors: David Idris Pritchard, Nottinghamshire (GB); Adele J. Horobin, Nottinghamshire (GB); Alan Brown, Nottinghamshire (GB)

Correspondence Address: JOHN S. PRATT, ESQ;KILPATRICK STOCKTON, LLP, 1100 PEACHTREE STREET, SUITE 2800, ATLANTA, GA, 30309, US

(73) Assignee: The Secretary of State for Defence, Salisbury, Wiltshire (GB)

(21) Appl. No.: 12/297,129

(22) PCT Filed: Apr. 13, 2007
(86) PCT No.: PCT/GB2007/050196

§ 371 Date: Aug. 11, 2009

(30) Foreign Application Priority Data
Apr. 13, 2006 (GB) 0607495.9

(51) Int. Cl. A61K 38/47; C12N 9/14

(52) U.S. Cl. 424/94.61; 435/195

(57) ABSTRACT

This invention relates to a polypeptide obtainable from insect larvae, such as those from Lucilia sericata, and which have activity as a nuclease in that they are able to degrade, denature, digest, cut or cleave nucleic acids such as DNA.

This invention relates to larval products. More particularly, the present invention relates to one or more polypeptides isolated from the larvae of Lucilia sericata (also named Phaenicia sericata) which polypeptides have the ability to degrade, to digest or to cut or cleave nucleic acids.

The benefits associated with the use of insect larvae or maggots in the healing of wounds were first observed and recognised centuries ago but it is only recently, especially with the increase in antibiotic resistant micro-organisms, that such use, termed “biosurgery”, has been readdressed and the renaissance of maggot therapy begun.¹⁰ The larvae currently in clinical use are those of the green bottle fly Lucilia sericata.

It is believed that larvae, or maggots, have a remarkable ability both to debride and to disinfect wounds. More recently it has been determined that this ability extends to the stimulation of tissue regeneration and to wound closure. Larvae are usable in the clinical setting to improve and to increase the rate of healing of many chronic wounds where conventional therapy such as antibiotics, antimicrobials bactericides, surgical debridement and wound drainage are unable to reduce or to stop the progressive tissue destruction.¹¹ Such chronic wounds originate from various conditions and include; diabetic foot ulcers, venous leg ulcers, infected surgical wounds, orthopaedic wounds, osteomyelitis and pressure sores.

The use of whole larvae or maggots is unpleasant to some patients and in an attempt to overcome this there has been a move towards the use of products obtained from the larvae rather than the larvae themselves. The constituents of the larval excretory/secretory (ES) products or the larval extracts are thought to be equally effective to the use of whole larvae. The constituents of the larval products are central to the way in which maggots promote wound healing with several mechanisms proposed to explain their actions. Debridement is thought to be partially achieved via the proteolytic action of constituent collagenase/s and serine protease/s present in the ES or extract which degrade the necrotic tissue into a form which is ingestible to the larvae and which act to remove the slough from the wound surface.^{12,13} Two antibacterial factors identified in the ES have shown activity against gram negative and gram positive bacteria, with one of these factors displaying significant activity against MRSA.¹⁴ The stimulation of extra-cellular matrix (ECM) remodelling and closure has also been proposed to be attributable to the action of one or more ‘chymotrypsin-like’ serine protease/s present in the ES which acts to degrade fibronectin into bioactive peptides. It is thought that these bioactive peptides are then able to stimulate adhesion and migration of fibroblasts¹⁸ and are possibly responsible for the acceleration in healing.¹⁵ The ES may also have an effect on fibroblast proliferation.

The present inventors have determined that the larval ES and extract both contain one or more polypeptides which are able to degrade, to denature, to digest or to cut or cleave nucleic acids. Preferably, the or each polypeptide comprises one or more of the sequences shown below.

Without wishing to be bound by theory the present inventors postulate that at least one of these polypeptides is or functions as a nuclease in that it degrades, denatures, digests, cleaves or cuts nucleic acids, especially DNA. In this respect it is believed that the polypeptides are nucleases in that they function as a nuclease even though at least some of them show no homology to known insect nucleases, as will be discussed further below.

The present inventors compared the homology of the polypeptide sequences having nuclease functions from larval ES or extracts to polypeptides in insect sequence databases and the closest homologies identified were to portions of insect ferritin proteins. Ferritins are globular protein complexes consisting of 24 protein subunits and are the main intracellular iron storage protein in both prokaryotes and eukaryotes, keeping the iron in a soluble and non-toxic form. Although a ferritin would not classically be described as a nuclease, it has been found²¹⁻²² that the iron released from ferritins in reaction media may act on nucleic acids, especially DNA, giving a result comparable to that of a DNase.

The polypeptides identified by the present inventors fit the definition of a nuclease in that they are capable of cleaving the phosphodiester bonds between the nucleotide subunits of nucleic acids or more specifically of DNA, they are able to catalyse the hydrolytic cleavage of phosphodiester linkages in the DNA backbone and thus are one type of nuclease. Hence, for the purposes of this application, the term nuclease or DNase will be used to describe the polypeptides of the present invention based on the observation of this activity being present in the polypeptides, regardless of whether the polypeptide would classically be described as a nuclease or DNase.

The present inventors have determined that the ES and extracts from the larvae of Lucilia sericata (also named Phaenicia sericata) comprise at least one nuclease. The nuclease may be used in the treatment of wounds, burns and the like or in the treatment or prophylaxis of infection. The nuclease may also be used in the treatment of cystic fibrosis, especially as an alternative to current DNAse therapies.

Accordingly, the present invention provides a nuclease isolated from insect larvae or a synthetic analogue or version thereof. Preferably, the nuclease comprises one or more of the polypeptide sequences given below:—

| | |
|---|---|
| LLEYLSMR | (SEQ ID NO: 1) |
| SFDDTLDMLK | (SEQ ID NO: 2) |
| SYEYLLLATHFNSYQK | (SEQ ID NO: 3) |
| SLGNPELPTEWLDLR | (SEQ ID NO: 4) |
| ELDASYQYLAMHK | (SEQ ID NO: 5) |
| VFVNSGTSLMDVR | (SEQ ID NO: 6) |
| ANFNWHESS | (SEQ ID NO: 7) |

It is possible that, due to the method used to obtain the above polypeptide sequence (mass spectroscopy, which randomly breaks peptide bonds), these polypeptides are the breakdown products of a longer polypeptide or protein, especially in view of the fact that they resolve as a single spot on a 2-D SDS-PAGE. Hence, the present invention also encompasses a polypeptide sequence comprising two or more of the polypeptide sequences identified above. Ideally, the present invention comprises a polypeptide sequence comprising all of the sequences identified above, optionally, in a single polypeptide chain. The two or more polypeptides need not be in the numerical order given. It is preferred that any such polypeptide has the above defined nuclease function.

Also included within the scope of the present invention are the nucleotide sequences encoding the polypeptides of SEQ ID NOs 1 to 7 and their use in the preparation of synthetic or recombinant polypeptides having the sequences given.

Preferably, the nuclease is suitable for use in the treatment or prophylaxis of infection or for the treatment of wounds. The nuclease may be used in a pharmaceutical composition, or incorporated into a dressing.

The term “wound” as used herein is intended to define any damage to the skin, epidermis or connective tissue whether by injury or by disease and as such is taken to include, but not to be limited to, cuts, punctures, surgical incisions, ulcers, pressure sores, burns including burns caused by heat, freezing, chemicals, electricity and radiation, dermal abrasion or assault, osteomyelitis and orthopaedic wounds. The wound may be infected. Additionally, the wound may be chronic or acute.

Preferably, the larvae are larvae from Lucilia sericata (also known as Phaenecia sericata).

The product may be a nuclease, and may be either a DNase, a RNase or a mixture thereof.

Ideally, the nuclease degrades both prokaryotic and eukaryotic nucleic acids, especially, both bacterial and mammalian nucleic acids. However, the nucleases of the invention may also be used in the treatment or prevention of viral replication.

The nuclease may be used to adjuvantise a conventional antibiotic in the treatment of infection, whether systemic or topical.

Nucleases, such as DNase enzymes, have been suggested to aid in wound debridement and have been incorporated into medicaments used to promote the debridement of chronic wounds. When used in the wound environment, DNases obtained from bovine pancreas have been shown to degrade the deoxyribonucleoproteins and deoxyribonucleic acid present in the necrotic tissue of chronic wounds.¹⁶ Refined extracellular products from a Lancefield group C strain of Streptococcus, Streptococcus equisimilis, form the constituents of the medicament Varidase®. Varidase® is a topical agent for the debridement of purulent wounds and contains a DNase component referred to as Streptodornase. Degradation of the extracellular DNA by streptodornase is thought to liquefy the pus, thereby allowing the free movement of leukocytes which clump in the presence of extracellular nucleoprotein and results in enhanced phagocytosis and wound healing.² However, it has been found that nucleases from larval sources, particularly those from Lucilia sericata are more robust in their resistance to the presence or absence of selected metal ions, and in their temperature-related activity.

The present inventors have found a metal ion dependent deoxyribonuclease (DNase) activity within ES and larval extract. This may contribute to wound debridement by degrading free DNA within non-viable tissue, thus reducing the viscosity of exudates or eschar. They have compared the DNase activity of the ES DNase with a commercial DNase I preparation and have found that ES DNase has a lower rate of reaction (Vₘₐₓ) than commercial DNase, when DNase substrate is non-limiting, but has a higher affinity (higher Kₘ) for DNA when the substrate becomes limiting. They have also found that ES DNases are more resistant to temperatures of between 20-40° C., a range relevant to the wound state in which a temperature gradient is present, where cold necrotic tissue will be at ambient temperature and the temperature of the other tissue is reduced when dressings are changed or when blood supply is compromised.¹⁷ In addition, the inventors have results showing that ES is less inhibited by ethylenediamintetra-acetic acid (EDTA) than commercial DNase and are more resilient to high Ca²⁺ and Na⁺ ion concentrations. These results indicate that ES DNase activity is more robust to changes in metal ion concentrations, compared with commercial preparations.

A nuclease activity has also been detected in extracts from larvae as will be described below.

The present invention will now be described, by way of example only, with reference to and as illustrated by the appended drawings of which

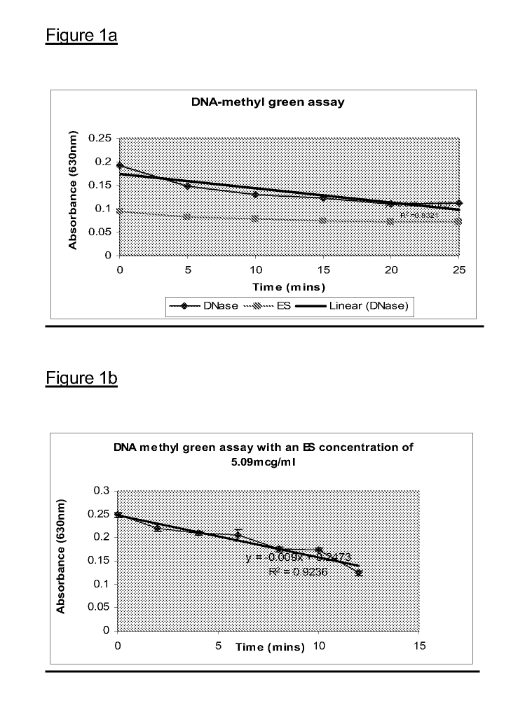

FIG. 1 is a pair of graphs showing the results of the DNA methyl green assays at high (FIG. 1a) and low (FIG. 1b) concentrations of larval ES;

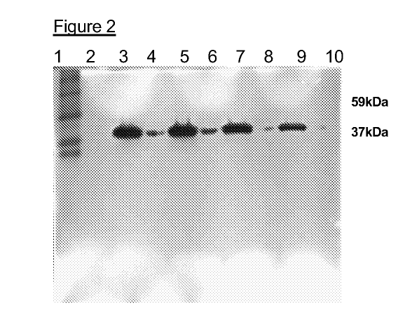

FIG. 2 is a photograph of a gel showing degradation of the DNA methyl green component of the gel by ES;

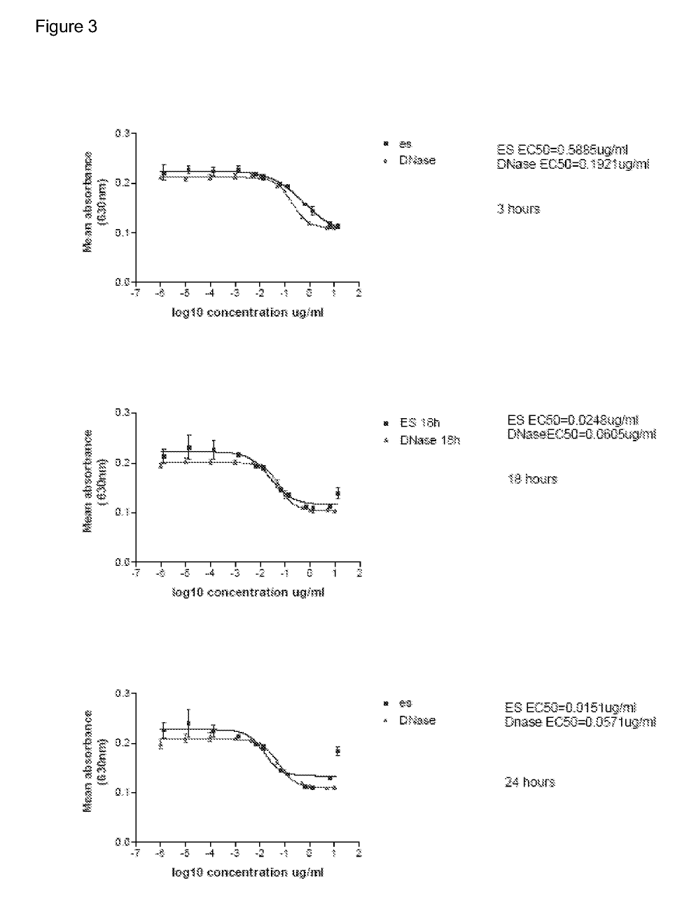

FIG. 3 is a series of dose dependency curves of ES and commercially-available DNase in relation to time at 3, 18 and 24 hours;

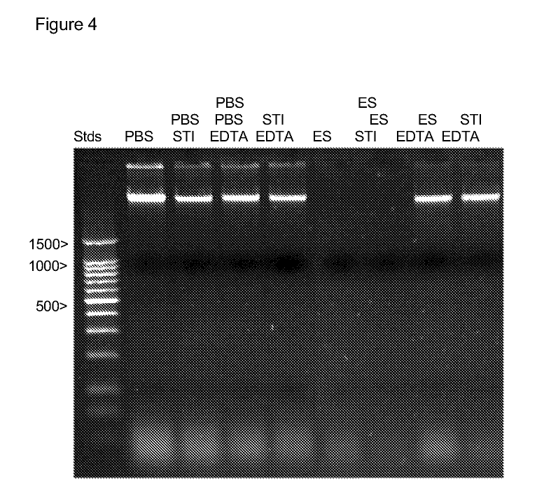

FIG. 4 is a photo of a gel showing the determination of DNase activity in the presence of EDTA and upon treatment with a soybean trypsin inhibitor (STI);

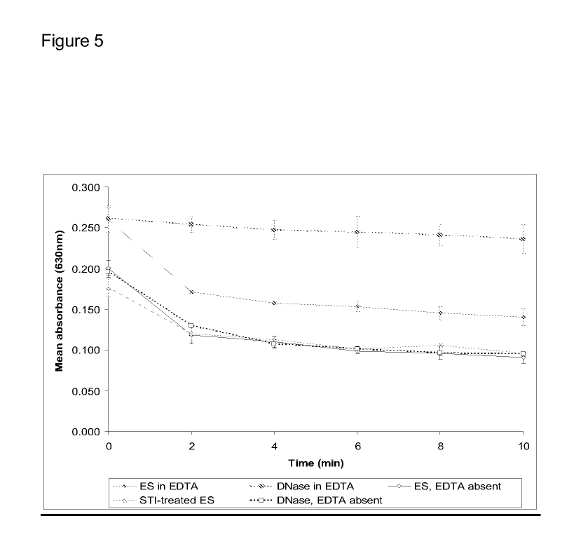

FIG. 5 is a is a graph showing DNA methyl green assay of the DNase activity of ES and DNase I in the presence of EDTA;

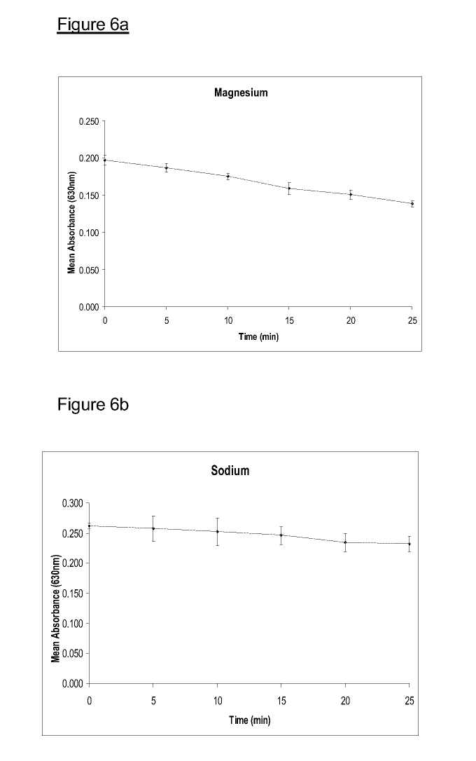

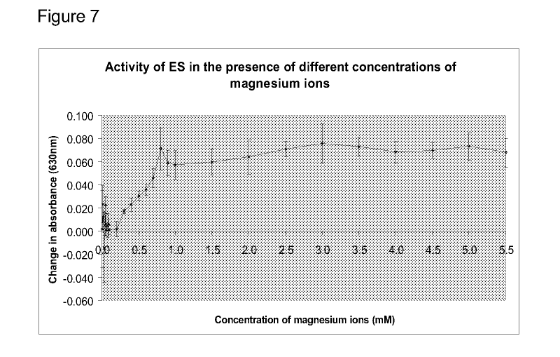

FIG. 6 is a graph which shows DNA methyl green assay with ES in the presence of 7.5 mM Mg²⁺ (FIG. 6a) and 7.5 mM Na⁺ (FIG. 6b);

FIG. 7 is a graph which shows the activity of ES in the presence of different concentrations of magnesium ions;

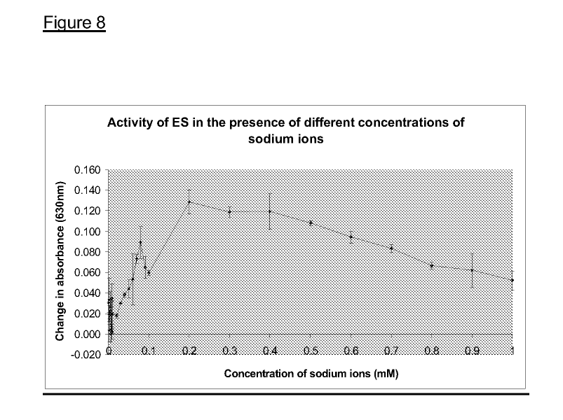

FIG. 8 is a graph which shows the activity of ES in the presence of different concentrations of sodium ions;

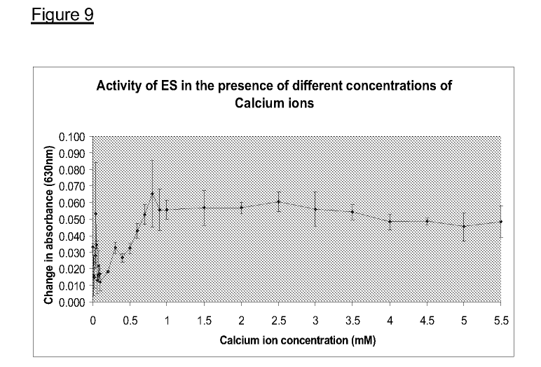

FIG. 9 is a graph which shows the activity of ES in the presence of different concentrations of calcium ions;

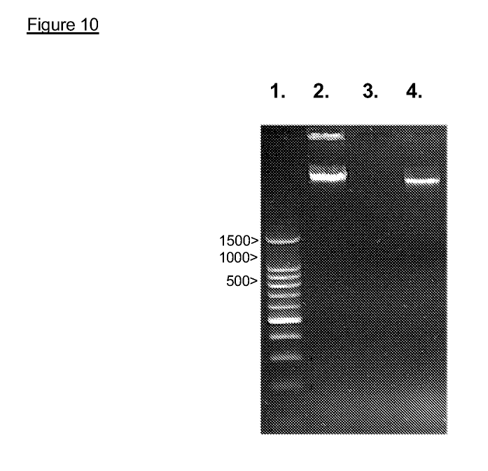

FIG. 10 is a photo of a gel showing shows Agarose gel electrophoresis of E. coli DNA;

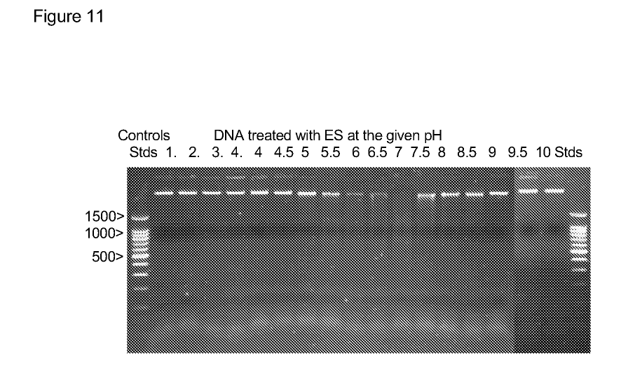

FIG. 11 shows Agarose gel electrophoresis of E. coli DNA (0.5 ng per lane) following 10 m incubation at 37° C. in the absence or presence of 0.02 μg/ml ES within a buffer of the stated pH;

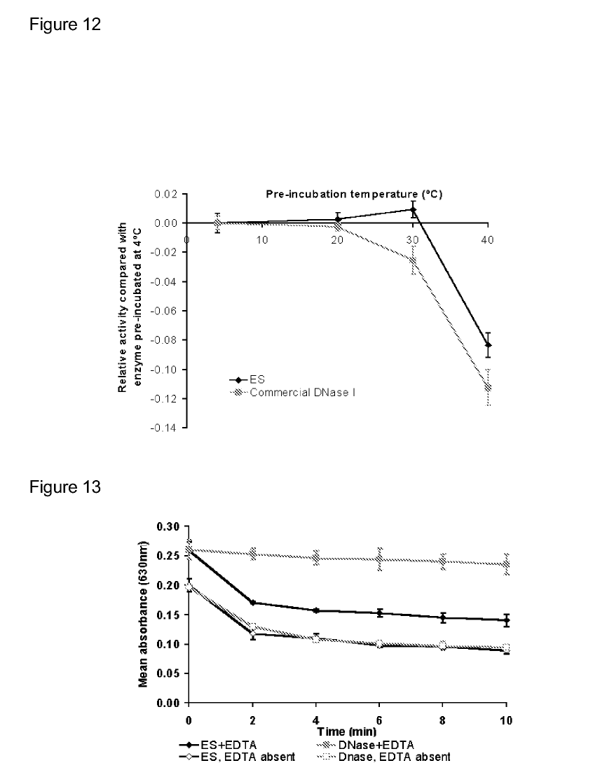

FIG. 12 is a graph showing shows degradation of DNA-methyl green complex, over 24 h at 37° C., by ES or commercial DNase that had been pre-exposed to the given temperature for 30 min and allowed to cool;

FIG. 13 is a graph showing degradation of DNA-methyl green complex, following exposure to ES or DNase for the time indicated, at 37° C., in the presence or absence of 5 mM EDTA;

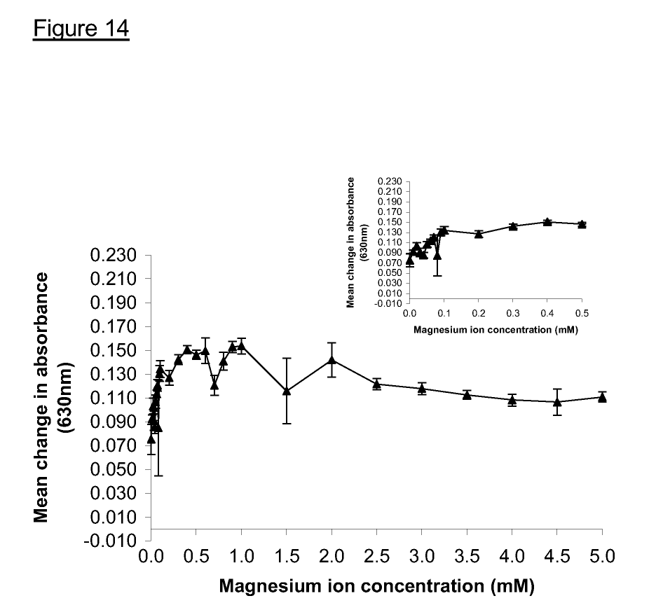

FIG. 14 is a graph which shows degradation of DNA methyl green following 19 h incubation, at 37° C., in the presence of 1 μg/ml ES and the magnesium ion concentration indicated;

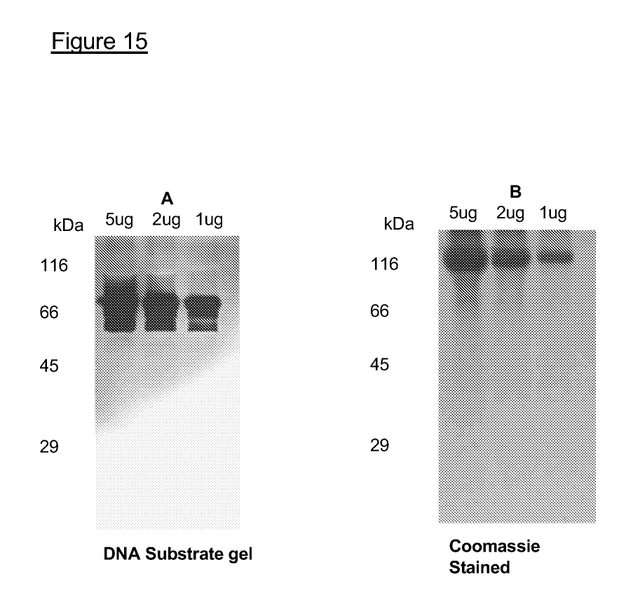

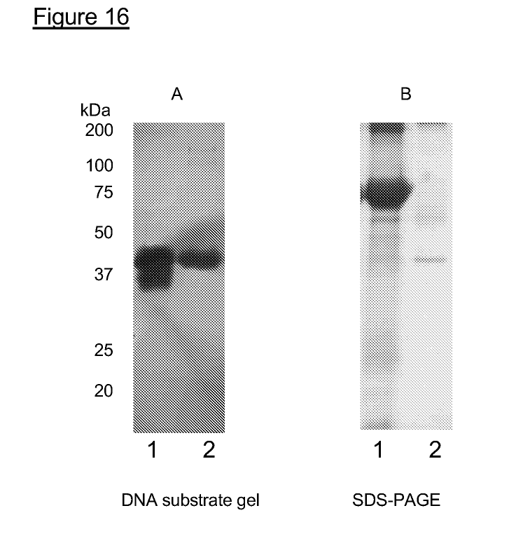

FIG. 15 is a photo of a gel showing DNA substrate (panel A) and protein (panel B) analysis of the DNase activity present in L. sericata extracts;

FIG. 16 is a photo of a gel showing DNA substrate (panel A) and protein (panel B) analysis of the DNase activity from L. sericata extract following purification on DNA cellulose,

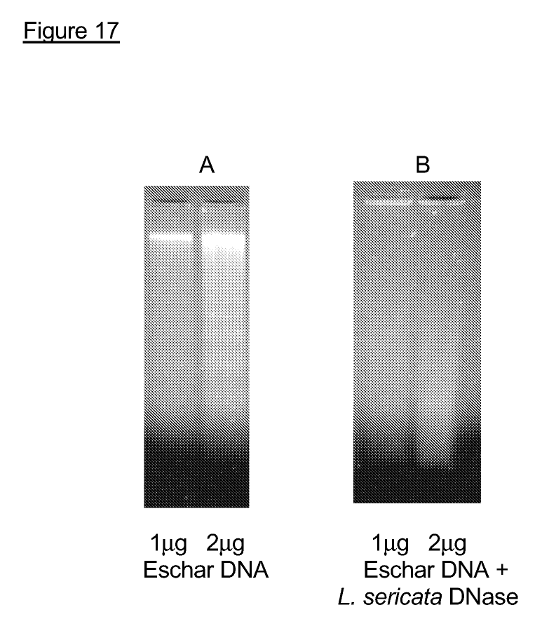

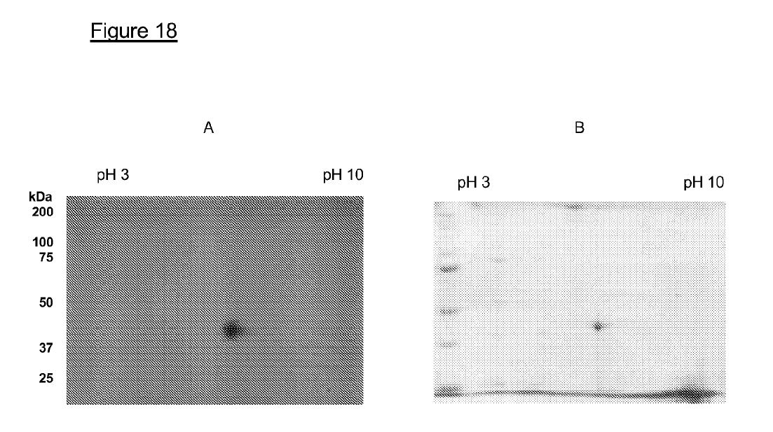

FIG. 17 is a photo of a gel showing Digestion of DNA from non-healing wound eschar by purified L. sericata DNase, and

FIG. 18 is a photo of a gel showing DNA substrate (panel A) and protein (panel B) analysis of the DNase activity from L. sericata extract following purification on DNA cellulose and 2-dimensional electrophoresis.

### EXAMPLE 1

The following experiments were carried out to identify and characterise a nuclease, especially a DNase from the ES.

### Collection of Lucilia Sericata Larval Excretory/Secretory Products

Approximately 300 freshly hatched Lucilia Sericata obtained from LarvE™, Surgical materials testing laboratory, Cardiff, UK, were washed repeatedly and their secretions (ES) collected, the process was carried out under aseptic conditions. 1 ml of sterile phosphate buffered saline (PBS) was added to the larvae and the larvae then left for 30 mins. Following this time the PBS and secretions were removed and the larvae left to recover for 30 minutes. This process was carried out around 4 times. The ES collected was then pooled for use.

Characterisation of Lucilia sericata Excretory/Secretory Products

The ES protein concentration in the PBS was measured using a Bio-Rad protein assay kit. (Hercules, Calif., U.S.A)¹

An FITC Casein assay was performed in order to determine ES proteinase activity. ES was diluted 1 in 20 with 0.1 mol L⁻¹ Tris-HCl buffer containing 5.3% FITC-casein conjugate, this was then incubated at 37° C. for 2 hours. Trichloroacetic acid 5% was then added in order to stop the reaction and left for 45 mins at room temperature. The protein precipitate formed was centrifuged to form a pellet and the resulting supernatant mixed 1 in 10 with 0.5 mol L⁻¹ Tris-HCl (pH 8.8). The fluorescence was measured at 485 nm excitation wavelength and 538 nm emission wavelength. The fluorescence detected from an ES blank sample was subtracted from the resultant fluorescence value.

### DNase Activity Assayed Using DNA and Methyl Green

DNA has a strong affinity for methyl green and forms a complex with the dye. DNase activity results in a loss of affinity of the DNA for methyl green and a colour change of the solution from green to colourless occurs. The activity of a standard DNase I enzyme and ES was compared. 1 mg/ml DNase was diluted 1 in 50 and 125 μl of this solution added to 1875 μl of 0.2 mg/ml DNA methyl green substrate solution. 125 μl of 162.3 μg/ml ES was also added to 1875 μl DNA-methyl green substrate solution. The resultant concentrations were therefore 1.28 μg/ml DNase and 10.14 μg/ml ES.

These solutions were then incubated in a water bath at 37° C. 100 μl aliquots were taken at 5 minute intervals for 25 minutes, and hourly for 5 hours (samples taken in triplicate at each time interval). These aliquots were added to 150 μl of sodium citrate in a 96 well plate. When all the samples were taken, the plate was left to stand overnight. The absorbance was then measured at 630 nm in an Anthos Lucy1 microplate luminometer.

This assay was then repeated at 2 minute intervals with varying concentrations of ES (5.09 μg/ml, 2.54 μg/ml, 1.27 μg/ml, 0.634 μg/ml, 0.317 μg/ml, 0.159 μg/ml ES) and 1.25 μg/ml DNase. The repetition was carried out due to apparent substrate exhaustion over the longer time period and with the higher concentration of ES.

### DNase Activity Assayed Via Electrophoresis in a DNA Methyl Green Containing Gel

The principle underlying this method is that the DNase enzymes migrate down the gel resulting in bands where the enzymes have degraded the DNA methyl green complex in the gel. Two gels were made; both were SDS polyacrylamide gels with a DNA methyl green concentration of 0.067 mg/ml. 20 μl samples of reducing concentrations of ES (10.14 μg/ml, 5.07 μg/ml, 2.54 μg/ml, 1.268 μg/ml) and DNase (10 μg/ml, 5 μg/ml, 2.5 μg/ml, 1.25 μg/ml) were produced via serial dilutions in PBS and water respectively. 20 μl of non-reducing sample buffer was added to each of these samples followed by incubation at 37° C. for 30 minutes. The DNase samples and ES samples were loaded onto different gels in addition to a pre-stained standard and a PBS/H₂O control. 0.1% SDS running buffer was used and a current applied. When the gels had run they were incubated with 2.5% TWEEN®, followed by washing in distilled water for 30 minutes and left overnight at 37° C. in 0.5M TRIS buffer containing 7.5 mM MgSO₄. Following this, the gels were stained with ethidium bromide and the results visualised under a transluminator.

### Dose Dependency Curves

In order to further characterise ES, dose dependency curves were created enabling the EC₅₀ value to be determined. ES was diluted to a concentration of 13.40 μg/ml in PBS, following this 1 in 10 dilutions in PBS were then performed. DNase was diluted to a concentration of 10 μg/ml in PBS, this was then also subject to 1 in 10 dilutions. 20 μl of each dilution was added to 180 μl of 0.2 mg/ml DNA methyl green in a 96 well plate (done in triplicate). Absorbance readings were then taken after 3, 18, and 24 hours at 630 nm in an Anthos Lucy1 microplate luminometer. The dose dependency curves were created by plots of log10 concentration (mcg/ml) against mean absorbance (630 nm) in the statistical analysis program, GraphPad Prism™.

### DNase Activity in the Presence of Inhibitors

### Electrophoresis of Samples in the Presence of a Soybean Trypsin Inhibitor and EDTA

Samples containing bacterial genomic DNA, and ES or DNase in the absence and presence of EDTA and a soybean trypsin inhibitor were loaded onto an agarose gel to observe the effects of these inhibitors on DNase activity. A soybean trypsin inhibitor (Sigma) was washed 4 times with PBS and each time centrifuged into a pellet and the PBS removed as the supernatant. On the 5^{th} washing, 500 μl of PBS were used to suspend the trypsin inhibitor and this was equally distributed between the samples to be treated. Samples of ES 162.3 μg/ml and PBS (control) were treated with the soybean trypsin inhibitor in order to remove any serine proteinases present. These samples were treated with STI 3 times in order to achieve maximal removal of serine proteinases, again following each treatment the solution was centrifuged and the ES/PBS removed as the supernatant. Following washing, these solutions were transferred to a fresh Eppendorf.

15.34 μl of 1.385 mg/ml bacterial genomic DNA was diluted to 0.1 mg/ml in 184.6 μl of either sterile PBS or 5 mM EDTA. 121.1 μg/ml DNase was also made up (10 μl of 1 mg/ml diluted in 72.6 μl of PBS.) In order to make up the samples, 50 μl of the appropriate one of these DNA solutions plus 3.125 μl of STI treated/untreated DNase/ES/PBS. 10 μl of loading buffer (Sigma) was added to each sample and the samples were incubated at 37° C. for 20 minutes and were then loaded onto 50 ml of 1.6% (w/v) agarose gel containing 5 μl of ethidium bromide.

Therefore, in the lanes in the gel were approximately 1 μg of DNA and effective concentrations of 7.56 μg/ml of DNase or 10.14 μg/ml of ES. The results were visualised in a transluminator.

### DNA Methyl Green Assay of ES and DNase Activity in the Presence of EDTA

125 μl of 81.15 μg/ml ES was added to both 1875 μl DNA methyl green (0.2 mg/ml) and DNA methyl green containing 5 mM EDTA. Final concentrations are therefore 5.07 μg/ml ES and 4.7 mM EDTA. DNase was diluted to 121.1 μg/ml, diluted 1 in 2 and added to 1875 μl of DNA methyl green and DNA methyl green containing EDTA. The activity of STI treated ES was also assayed in the same way, in the presence of or in the absence of EDTA. These samples were then incubated in a water bath at 37° C. 100 μl samples were taken every 2 minutes (in triplicate) and added to 150 μl of 0.083M sodium citrate in a 96 well plate. The plates were left to stand overnight and the absorbance then read at 630 nm in an Anthos Lucy1 microplate luminometer.

### Activity of ES in the Presence of Various Metal Ions

ES was diluted 1 in 10 with sterile PBS and EDTA giving resultant concentrations of 5 mM EDTA and 16.23 μg/ml ES. 125 μl of this solution was then added to 1875 μl of 0.2 mg/ml DNA methyl green containing 7.5 mM of a particular ion (Copper, zinc, magnesium, sodium, nickel, calcium). Final concentrations were therefore, 0.3125 mM EDTA and 1.014 μg/ml ES. 100 μl samples were taken every 2 minutes and added to 150 μl of sodium citrate 0.083M in a 96 well plate (in triplicate), the remainder of the sample being kept at 37° C. The plates were again left to stand overnight at room temperature and the absorbance readings taken at 630 nm in an Anthos Lucy1 microplate luminometer.

In order to estimate the optimum ion concentrations of Mg²⁺, Ca²⁺ and Na⁺ for ES, an array of concentrations were made up from three 0.2 mg/ml DNA methyl green stock solutions containing 10 mM Mg²⁺, 10 mM Ca²⁺ and 2 mM Na⁺. (Concentrations were achieved by diluting in non ion containing DNA methyl green.)

10 μl of 0.2 μg/ml ES was added to 90 μl of the ion containing DNA methyl green concentrations. (ES was diluted from neat in 0.5M Tris, pH 7.5) The final concentration of ES in DNA methyl green was therefore 0.02 μg/ml. Triplicates of each concentration were made up in order to give an average absorbance. Blanks were also made up as the ions altered the colour of the DNA methyl green and therefore the absorbance. The absorbance readings of both the actual plates and the blanks were therefore taken at 630 nm in an Anthos Lucy1 microplate luminometer. The change in absorbance between the blank and result plate was calculated and plotted against the ion concentration.

### Results

### Determination of the DNase Activity of Larval Excretory/Secretory Product (ES)

The DNA methyl green assays carried out demonstrated the DNase activity of the ES due to the significant degradation of the DNA methyl green complex, seen statistically as the reduction in absorbance over time illustrated in FIG. 1. A clear reduction in the absorbance occurs for both the DNase and the ES samples, indicating that ES does indeed display DNase activity. There is a significant flattening of the curves produced, particularly for ES, possibly thought to be due to substrate exhaustion due to the high concentration of ES used or because the time interval for sampling was too long. Later it was also considered possible that the sodium citrate used to stop the degradation of the DNA methyl green complex and DNase activity was not completely inhibiting the reaction. Therefore when the plates were left to stand over night, the reaction was continuing. The results are shown in FIG. 1a which shows DNA methyl green assay: 1.28 μg/ml DNase and 10.14 μg/ml ES in 0.2 mg/ml DNA methyl green substrate solutions. The solutions were incubated for 25 mins and 100 μl samples taken (in triplicate) at 5 min intervals. The samples were added to 150 μl of sodium citrate (in a 96 well plate) to stop the reaction and the absorbance readings taken the following day, these readings are displayed in FIG. 1a.

The experiment was therefore repeated with sampling at 2 minute intervals and varying concentrations of ES. At the concentrations of ES below 5.09 μg/ml the curves produced demonstrated minimal DNase activity but at 5.09 μg/ml a linear curve was produced demonstrating the significant DNase activity of ES at this concentration. The results are shown in FIG. 1b DNA methyl green assay: 5.09 μg/ml ES in 0.2 mg/ml DNA methyl green substrate solution. The solution was incubated at 37° C. and 100 μl samples taken every 2 minutes. The samples were added to 150 μl of sodium citrate (in a 96 well plate) to stop the reaction and the absorbance readings taken the following day. The readings are displayed in FIG. 1b.

This DNase activity was confirmed by sodium dodecyl sulphate-polyacrylamide gel electrophoresis (SDS-PAGE), where this gel contained 0.067 mg/ml DNA methyl green. 2 gels were used, 1 loaded with reducing concentrations of DNase and 1 with reducing concentrations of ES. The ES displayed definite DNase activity visible as the degradation of the DNA methyl green in the gel. It also appeared that there was a greater level of degradation at the lower concentrations of ES but this would need to be repeated to assess the viability of this finding. The results are shown in FIG. 2 where FIG. 2-Degradation of the DNA methyl green component of the gel by ES. Lane 1-Pre-stained Standard, Lane 2-PBS control, Lane 3-1.268 μg/ml ES, Lane 5-2.54 μg/ml ES, Lane 7-5.07 μg/ml ES, Lane 9-10.14 μg/ml ES, and Lanes 4, 6, 8 and 10 are empty.

### Dose Dependency Curve

Serial dilutions of ES (13.40 μg/ml) and DNase (10 μg/ml) solutions, the addition of 20 μl of each dilution to 180 μl of DNA methyl green in a 96 well plate and the incubation of these well plates at 37° C. allowed for the construction of 2 dose dependency curves. Absorbance readings were taken at 3, 18 and 24 hours and the curve constructed from the readings at 24 hours. (See FIG. 3) From the dose dependency curves, the EC₅₀ values were also able to be calculated using GraphPad Prism™. The EC₅₀ values of ES and DNase were found to be 0.5885 μg/ml and 0.1921 μg/ml at 3 hours, 0.0248 μg/ml and 0.0605 μg/ml at 18 hours and 0.0151 μg/ml and 0.0571 μg/ml at 24 hours, respectively, thereby indicating that the DNase used is 3 times more potent than ES. The results are shown in FIG. 3.

### Ion Dependencies of ES

Various samples of bacterial genomic DNA and ES or DNase, both with and without the ion chelator EDTA, treated and untreated with a trypsin inhibitor, were used and loaded onto an agarose gel. This was done in order to determine if the activity of the DNase enzymes was inhibited by EDTA and therefore seen to be ion dependant. Treatment with the trypsin inhibitor should remove any trypsin and chymotrypsin enzymes, thereby identifying if DNase activity is independent of these enzymes. The control samples containing PBS exhibited no DNA degradation as expected, the undegraded DNA appears as the same distinct band (lanes 2-5). The results obtained for ES containing samples indicated that the DNase activity observed is not due to chymotrypsin activity (lane 8) as the DNA is still degraded in the absence of this enzyme. It is also clear that DNase activity is inhibited by EDTA as there is no DNA degradation in lane 9, therefore indicating that the activity of ES is ion dependant. The DNase samples (lanes 11 and 12) both in the absence and presence of EDTA display no DNase activity. The DNase may therefore be inactive against bacterial genomic DNA as it would be expected to degrade the DNA when in the absence of EDTA. The results are shown in FIG. 4, Determination of DNase activity in the presence of EDTA and upon treatment with a soybean trypsin inhibitor (STI)

Lane 1-DNA ladder; Lane 2-DNA+untreated PBS; Lane 3-DNA+STI treated PBS; Lane 4-DNA+untreated PBS+EDTA; Lane 5-DNA+STI treated PBS+EDTA; Lane 6-Empty; Lane 7-DNA+untreated ES; Lane 8-DNA+STI treated ES; Lane 9-DNA+untreated ES+EDTA; Lane 10-DNA+STI treated ES+EDTA; Lane 11-DNA+untreated DNase; Lane 12-DNA+untreated DNase+EDTA.

### DNase Activity of ES and DNase I in the Presence of EDTA

DNase and ES have both been shown to be inhibited by EDTA but DNase more significantly so, with its activity almost abolished in the presence of EDTA. FIG. 5 illustrates the results of this DNA methyl green assay whereby DNase activity is illustrated by the resultant reduction in absorbance as the DNA methyl green complex is degraded. In the absence of EDTA, ES and DNase have relatively similar activity at these concentrations (5.07 μg/ml and 3.78 μg/ml respectively). The results are shown in FIG. 5 which is a graph showing DNA methyl green assay of the DNase activity of ES and DNase I in the presence of EDTA.

### The Effect of Different Metal Ions on the DNase Activity of ES

ES activity was monitored in the presence of 7.5 mM copper, zinc, magnesium, sodium, nickel and calcium ions. Magnesium ions were shown to exert a positive influence on ES DNase activity with a possible sodium influence also, although the linear curve produced was not as convincing as that produced in the magnesium assay (FIGS. 6a and 6b). FIG. 6a shows DNA methyl green assay with ES in the presence of 7.5 mM Mg²⁺. Magnesium is seen to activate ES as is apparent through the degradation of the DNA methyl green complex and the consequential reduction in absorbance. FIG. 6b shows DNA methyl green assay with ES in the presence of 7.5 mM Na⁺. It appears that Na⁺ also activates ES but not as significantly as Mg⁺.

Subsequently, Mg²⁺, Ca²⁺ and Na⁺ were all found to stimulate ES DNase activity. Increasing the concentration of Ca²⁺ and Mg²⁺ ions was found to cause an increase in ES activity up to a point, after which the activity remained at a relatively constant level. The optimum Mg²⁺ concentration was found to be 3 mM and the optimum Ca²⁺ concentration was 0.9 mM. ES activity in the presence of Na⁺ ions was found to increase rapidly with optimum activity at 0.2 mM after which the activity of ES steadily decreased. The activity of ES in the presence of the these various cations was determined by using, ‘blanks,’ of each ion concentration in DNA methyl green which does not contain ES and subtracting these absorbance values from those of the actual concentrations containing ES, following incubation for 24 hours. The results are shown in FIGS. 7-9.

### Discussion

Lucilia sericata excretory/secretory product has displayed definite DNase activity independent of previously identified chymotrypsin-like serine proteases, thereby a new DNase component to the larval secretions has been identified. This therefore indicates a further process by which the maggot acts to promote the healing of chronic wounds. Previous studies have noted that extracellular nucleoprotein in the wound site results in the aggregation and clumping of leukocytes, which contributes to poor wound drainage. An improvement in wound healing is achieved upon DNA degradation, liquefaction of pus with the resultant improved movement of leukocytes and phagocytosis.²

The experimental procedure used allowed colorimetric determination and confirmation of DNase I activity using a DNA-methyl green substrate.³ Of greater relevance to the wound site it was also found that the DNase present in ES is also able to degrade bacterial genomic DNA leading to the possibility that DNase activity, as well as aiding in wound debridement, may have a role in the antibacterial effect of the maggot which has previously been ascribed to protease activity⁴ and ingestion and destruction of bacteria by the maggot.⁵ The larval DNase displayed metal ion dependant catalysis similar to that observed in other DNases. The activity of the DNase excreted/secreted by Lucilia sericata has been shown here to be stimulated by magnesium, sodium and calcium ions. Activity appeared to be stimulated to the greatest extent by Mg²⁺, followed by Ca²⁺ followed by Na⁺. When compared to the tolerance of the streptodornase component of Varidase®, a previously marketed product for wound healing, the DNase activity of streptodornase appears to be far less capable in the presence of cations than the larval DNase. Magnesium stimulates DNase activity at low concentrations only (optimum 0.06 mM compared to larval DNase of 3 mM), after which activity rapidly deteriorates. Ca²⁺ and Na⁺ are inhibitory at all concentrations except when used in combination with Mg²⁺.² Ion concentrations in the chronic wound is an area which remains uninvestigated but the resilience of larval DNase to an extremely wide range of ion concentrations and its continued activity are a definite advantage for its efficacy in the wound environment.

A problem with the results obtained when investigating the activity of DNase over the wide range of Na⁺, Mg²⁺ and Ca²⁺ concentrations is that the blanks used in order to calculate the change in absorbance should ideally have been incubated over the same time period as the experimental samples. The absorbance readings for the blanks were taken immediately meaning that if the DNA methyl green complex displayed any instability due to the presence of the ions this will not have been taken into account when calculating the change in absorbance and leaves room for error. This experiment should therefore be repeated with the blanks incubated over the same time period as the experimental plates for confirmation of these findings.

The DNase activity of larval ES was also noted to be more resilient in the presence of EDTA than the standard DNase used, the activity of the standard DNase was almost abolished in the presence of the ion chelator whereas the larval DNase retained significant activity.

Larval DNase activity in the wound may aid in the debridement process, contribute to the antibacterial effect of maggots in the wound environment and it may also be hypothesized that the degradation of bacterial DNA into oligonucleotides could also act to modulate the immune system. It is known that components of the immune system respond to the contents of bacterial cells. It has also been found that sequences of bacterial DNA can have an immunostimulatory effect on certain immune cells, in particular dendritic cells and macrophages but also B cells.⁶ Bacterial CpG DNA is a pathogen associated molecular pattern (PAMP). A PAMP is a pattern shared by many pathogens but is not expressed by the host, these therefore act as important stimuli for the innate immune system. CpG DNA is immunostimulatory and has been shown to activate Toll 9 receptors thereby initiating cellular responses.⁷ The degradation of bacterial DNA by larval DNase could result in such immunostimulatory oligonucleotides of bacterial DNA. CPG-ODN activation of Toll-like receptors results in a protective reaction whereby reactive nitrogen and oxygen intermediate molecules, antimicrobial peptides, adhesion molecules, cytokines (TNFα, IL-12, p40 and IL-6) and acute-phase proteins are expressed.^{6,8} It has also been shown that the activation of toll-like receptors in dendritic cells and macrophages triggers the expression of co-stimulatory molecules such as CD40 and CD86 which interact with CD28 on T lymphocytes therefore playing an integral role in antigen presentation and stimulation of the adaptive immune system.^{8,9} Therefore it is possible that if larval DNase degraded bacterial genomic DNA into oligonucleotides with a central, unmethylated, cytosine-guanosine core, this may lead to cytokine release and activation of the immune system which would be a further effect elicited by the maggots aiding the healing process.

The ion experiment should also be repeated using the appropriate controls and could also be carried out using different combinations of metal ions. Locke and Carpenter² found that the DNase activity of the streptodornase component of Varidase® was greatest in the presence of equal concentrations of magnesium sulphate and calcium chloride and hypothesized that the streptodornase enzyme has 2 binding sites, this is an area which could be investigated for larval DNase. It would also be interesting to investigate the effects of larval DNase degraded, bacterial DNA on different cell types such as dendritic cells, macrophages and B lymphocytes in order to assess if the oligonucleotides produced may have a modulatory role.

Overall, these initial studies and the confirmed DNase activity of larval excretory/secretory product has introduced further possible mechanisms which add to the growing explanations of how the maggot so successfully manages to debride, cleanse and promote the healing of chronic wounds. This DNase activity has been shown to be cation dependant, displayed greater resilience and maintained DNase activity in the presence of the ion chelator EDTA than a standard DNase, and showed greater activity in the presence of increasing ion concentrations than that of streptodornase which was the DNase component of Varidase® (a streptokinase-streptodornase medicament) used for the cleansing of purulent wounds. It is therefore clear that this identified DNase activity is another crucial addition to the growing repertoire of ES actions in the promotion of wound healing.

### EXAMPLE 2

### Deoxyribonuclease (DNase) Activity within L. sericata ES

As shown in FIGS. 2 and 10, the present inventors have identified a DNase activity within ES. FIG. 2 shows Electrophoresis of ES under native conditions within a DNA/methyl green substrate polyacrylamide gel. The gel is counterstained with ethidium bromide and the dark area in lane 3 indicates where DNA has been digested by a nuclease activity in maggot secretions 1. Pre-stained standards (molecular weights indicated in kDa). 2. Buffer control. 3. ES (13 ng). FIG. 10 shows Agarose gel electrophoresis of E. coli DNA. Bands of DNA stained with ethidium bromide. 1. 100 bp standards (number of base pairs indicated). 2. E. coli DNA (1 μg) alone. 3. E. coli DNA (1 μg)+ES (7.5 μg/ml). 4. E. coli DNA (1 μg)+ES (7.5 μg/ml)+5 mM EDTA.

This is interesting because DNases may contribute to wound debridement by degrading free DNA within non-viable tissue, thus reducing the viscosity of exudate. It works best at neutral pH (FIG. 11) and is inhibited by ethylenediaminetetra-acetic acid (EDTA) (FIG. 4), indicating that its activity is metal ion-dependent. FIG. 11 shows Agarose gel electrophoresis of E. coli DNA (0.5 ng per lane) following 10 m incubation at 37° C. in the absence or presence of 0.02 μg/ml ES within a buffer of the stated pH. Bands of DNA stained with ethidium bromide. Controls: 1. DNA alone, untreated; 2., 3., 4. DNA within buffer pH4.0, 7.0 or 10 respectively, in the absence of ES. Stds refer to 100 bp standards (number of base pairs indicated). DNase activity occurs within a range of pH5.0-8.5, with optimal activity at pH7.0. FIG. 4 shows Agarose gel electrophoresis of E. coli DNA (1 μg per lane) following 20 m incubation at 37° C. in the presence or absence of 7.56 μg/ml ES (untreated or pre-exposed 3× to an excess of soybean trypsin inhibitor (STI) immobilised on cross-linked 4% beaded agarose), 5 mM EDTA or PBS (untreated or pre-exposed 3× to an excess of immobilised STI), at an equivalent volume as ES. Bands of DNA stained with ethidium bromide. Stds refer to 100 bp standards (number of base pairs indicated). DNase activity of ES is unaffected by STI treatment and may actually be enhanced (note the reduction of the faint band towards the bottom of the gel, indicating that removal of serine proteinase activity in ES results in enhanced degradation of small DNA fragments. It is not a serine proteinase as pre-exposure of ES to immobilised soybean trypsin inhibitor (STI) (removes serine proteinase activity) does not inhibit it (FIG. 4). In fact, STI treatment may result in a slight enhancement of activity. We then went on to compare the DNase activity in ES to a commercial DNase I preparation. FIG. 12 shows degradation of DNA-methyl green complex, over 24 h at 37° C., by ES or commercial DNase that had been pre-exposed to the given temperature for 30 min and allowed to cool. Results expressed as mean level of DNase activity, in comparison to an ES or DNase preparation that had been pre-incubated at 4° C., ±1 SD (n=3). As shown in FIG. 12, ES DNases are more resistant to temperatures between 20-40° C., a range relevant to the wound state. In addition, we have results showing that ES DNase is less inhibited by ethylenediaminetetra-acetic acid (EDTA) than commercial DNase (FIG. 13). FIG. 13 shows degradation of DNA-methyl green complex, following exposure to ES or DNase for the time indicated, at 37° C., in the presence or absence of 5 mM EDTA. Decrease in absorbance at 630 nm indicates degradation of the complex. Results shown as mean absorbance ±1 SD (n=3). By comparison with a relevant publication, is also less inhibited by high Mg²⁺ concentrations than Varidase, a commercial debridement preparation containing Streptokinase/Streptodornase (FIG. 14). FIG. 14 shows degradation of DNA methyl green following 19 h incubation, at 37° C., in the presence of 1 μg/ml ES and the magnesium ion concentration indicated. Results are expressed as the change in absorbance at 630 nm wavelength following completion of the incubation period (measurements taken at 0 h−measurements taken at 19 h). The greater the change in absorbance, the greater the degradation of the DNA methyl green complex. As shown in the insert of FIG. 14 (which shows an enlargement of the low ion concentration region), low Mg²⁺ concentrations slightly increase ES DNase activity. From 0.1 mM upwards, the ion concentration has little effect, with only a slight decrease in activity observed at the higher ion concentrations. Compare these findings with those of Locke et al (2002)² in FIG. 1, where Varidase activity is strongly inhibited at Mg²⁺ concentrations above 0.06 mM.

The present inventors also have preliminary results suggesting that ES DNases are more resilient to high Ca²⁺ and Na⁺ ion concentrations. These results indicate that ES DNase activity is more robust to changes in metal ion concentrations, compared with commercial preparations.

### EXAMPLE 3

The following experiments were carried out to identify and characterise a nuclease, especially a DNase from the larval extract.

### Preparation of Larval Extract

3^{rd} instar larvae of Lucilia sericata are homogenated. The homogenate is diluted in a buffer, such as phosphate buffered saline, and centrifuged to extract the soluble proteins. The supernatant is aspirated and used as the maggot extract.

### Detection of DNase Activity

DNase activity is detected using a SDS-PAGE substrate gel method in which calf thymus DNA is incorporated into the resolving gel at a concentration of 4 mg/ml. 3^{rd} instar maggot extracts prepared as above are pre-incubated in non-reducing sample buffer (0.1 M Tris-HCl, 10% Glycerol, 4% SDS, 0.04% Bromophenol Blue, pH 6.8.) for 30 min at 37° C. prior to loading onto the substrate gel. Following electrophoresis (20 mA/gel) gels were washed with 2.5% Triton (30 mins) followed by water (30 min).

To allow the DNA to be digested the gels were incubated overnight at 37° C. in phosphate buffered saline (PBS) plus 7.5 mM MgCl₂. Finally, DNase activity was visualised by staining with 5 μg/ml ethidium bromide and viewing on a UV transilluminator (FIG. 15A).

### Protein Detection

Protein was detected in SDS-PAGE gels by staining with 0.1% Coomassie Brilliant Blue R250 followed by destaining with 25% methanol, 10% acetic acid until protein bands were visualised (FIG. 15B).

As shown in FIG. 15 DNA substrate (panel A) and protein (panel B) analysis of the DNase activity present in L. sericata extracts. DNase activity typically resolves between approximately 35-45 kDa (panel A). However, very little corresponding protein is observed in this region (panel B). Similar activities can be demonstrated in L. sericata secretions.

### Purification of DNase Activity

DNase purification was attempted as described by Locke et al¹⁹. 3.5 g of 3^{rd} instar maggots prepared as above were extracted into 15 ml of low salt buffer (50 mM NaCl, 1 mM EDTA, 1 mM DTT, 10% Glycerol, 0.1 mg/ml BSA, 20 mM Tris, pH 8.1), centrifuged (13000 g, 10 min), and the supernatant passed through a 22 μM filter. A DNA cellulose column (Amersham) was equilibrated with low salt buffer prior to the application of maggot extract. Following application the column was washed with low salt buffer until the Abs at 280 nm of the wash buffer was zero. Bound protein was eluted with high salt buffer (2M NaCl, 1 mM EDTA, 1 mM DTT, 10% Glycerol, 0.1 mg/ml BSA, 20 mM Tris, pH 8.1), 1 ml fractions collected, their Abs measured at 280 nm and the protein peak eluted dialysed against PBS. The starting material and eluted protein were analysed for DNase activity by DNA substrate gel analysis (FIG. 16A) and protein content by SDS-PAGE (FIG. 16B).

As shown in FIG. 16, DNA substrate (panel A) and protein (panel B) analysis of the DNase activity from L. sericata extract following purification on DNA cellulose. Following elution from the column DNAse activity resolved at approximately 45 kDa (panel A) and corresponded to a protein band of the same molecular mass following Coomassie staining (panel B).

Wound DNA Digestion by Purified L. sericata DNase

Genomic DNA from non-healing wound eschar was prepared using Sigma's Genomic DNA prep kit. 1 and 2 μg of DNA purified from L. sericata extract on DNA-cellulose was incubated with approx 0.5 μg of purified DNase for 1 hr at 37° C. Digestion products were run on a 1% agarose gel (FIG. 17) and detected by ethidium bromide staining as described previously. FIG. 17 shows digestion of DNA from non-healing wound eschar by purified L. sericata DNase.

2-dimensional gel analysis of purified L. sericata DNase

Approximately 100 μg of DNase activity eluting from a DNA-cellulose column was applied to a first dimension isoelectro-focusing strip containing an immobilized pH gradient (3-10). The strip was focused using a Biorad Protein IEF kit according to the manufacturers' instructions. After focusing the DNase activity and protein profile of the strip was analysed using a DNA substrate gel (FIG. 18A) and SDS-PAGE (FIG. 18B) under non-reducing conditions as described previously. FIG. 18 shows the result of DNA substrate (panel A) and protein (panel B) analysis of the DNase activity from L. sericata extract following purification on DNA cellulose and 2-dimensional electrophoresis. DNase activity was again associated with a protein of approximately 45 kDa (panel A) and corresponded to a protein spot of the same molecular mass following Coomassie staining (panel B). This spot was sequenced by mass spectrometry with the following results.

### Sequence Analysis

7 peptide sequences were obtained following mass spectrometry:

| | |
|---|---|
| LLEYLSMR | (SEQ ID NO: 1) |
| SFDDTLDMLK | (SEQ ID NO: 2) |
| SYEYLLLATHFNSYQK | (SEQ ID NO: 3) |
| SLGNPELPTEWLDLR | (SEQ ID NO: 4) |
| ELDASYQYLAMHK | (SEQ ID NO: 5) |
| VFVNSGTSLMDVR | (SEQ ID NO: 6) |
| ANFNWHESS | (SEQ ID NO: 7) |

LLEYLSMR (SEQ ID NO:1) and SLGNPELPTEWLDLR (SEQ ID NO:4) have high homologies to a portion of the ferritin heavy chain from the tsetse fly Glossina morsitans.

| | |
|---|---|
| L. sericata | ------------------------SLGNPELPTSWLDLR----------- |
| Glossina morsitans | MMKLIVTLCILAVG [symbol] QIV [symbol] GEMKCSIGNPELPTEWI [symbol] LRGECL [symbol] AMPDQI |
| | *:*********:*** |
| L. sericata | --------------------------------------------LLEYLS |
| Glossina morsitans | Q [symbol] EIDASYTYLAMGA [symbol] FSRDTINRPGFAEHFKAAKEE [symbol] Q [symbol] GARLIBYLS |
| | *:**** |
| L. sericata | MR------------------------------------------------ |
| Glossina morsitans | MRGQLTDDVTDL [symbol] MVPTVSKHEWSSGTEAL [symbol] DALRLET [symbol] VTKS [symbol] RKL [symbol] QT |
| | ** |
| L. sericata | -------------------------------------------------- |
| Glossina morsitans | CERKMNYY [symbol] LVDWLTGVYLEEQLHGQRDLAGKISTLKKMMDNBGGLGEEL |
| L. sericata | ----- |
| Glossina morsitans | FOKEL |

[symbol]

indicates data missing or illegible when filed

SYEYLLLATHFNSYQK (SEQ ID NO:3) has very high homology to a portion of the ferritin light chain from the tsetse fly Glossina morsitans and the fruit fly Drosophila

| | |
|---|---|
| L.sericata | -------------------------------------------------- |
| Glossina morsitans | MEFLIFVAL [symbol] ASS--CVLL [symbol] ASSVCHNMVVRACSTSTLSGFSIC [symbol] ARYGG |
| Drosphila | M [symbol] LLVAFALIA [symbol] LGALAG [symbol] --EE [symbol] CHNSVVTACSSSTSSGNSICKAR [symbol] AG |
| L.sericata | ------------------SYE [symbol] YLL [symbol] AT [symbol] FNSYQK--------------- |
| Glossina morsitans | IS [symbol] YEPELQAYINSHLTKSYEYLL [symbol] ATHFNSYQ [symbol] NRPGPQKLYQSLSD [symbol] S |
| Drosphila | IEMYEPEVQAYINSQLTKSYEYLLEATHFNSYQ [symbol] NRPGPQKLYQGLSD [symbol] S |
| | **************** |
| L.sericata | -------------------------------------------------- |
| Glossina morsitans | SLDTI [symbol] M [symbol] KQ [symbol] TR [symbol] SSKADFNTRHESPASVSTQQQRLEVSEL [symbol] S [symbol] AWALD |
| Drosphila | SDDSIAL [symbol] KQITKFSGIVDFNTRHESPASVSTQRFTLEVSEL [symbol] SEALALD |
| L.sericata | -------------------------------------------------- |
| Glossina morsitans | NEKQLTTGAPHV [symbol] TQSL [symbol] AAR---D [symbol] ETAQYIEEKFLGSQAETIPKLSG [symbol] |
| Drosphila | NEDQLATGATHIRTRAIHATER--DEEMA [symbol] YMEEEYLGRQADSVPKLSG [symbol] |
| L.sericata | --------------------------- |
| Glossina morsitans | ANELAKLM [symbol] QPDEELAYYLFDEYLQEQ |
| Drosphila | ANELANLMPVPDESL [symbol] IYLFDEYLQEQ |

[symbol]

indicates data missing or illegible when filed

SFDDTLDMLK (SEQ ID NO:2) currently has no strong homology to anything in the database used²², the closest match found is to a DNA methylase.

ELDASYQYLAMHK (SEQ ID NO:5), VFVNSGTSLMDVR (SEQ ID NO:6) and ANFNVVHESS (SEQ ID NO:7) appear to have no convincing homologies with any known proteins on the data bases including known nuclease sequences and would therefore appear to represent novel nuclease sequence that are yet to be identified or to be added to insect protein sequence databases.

### REFERENCES

1) Bradford M. A rapid and sensitive method for the quantification of microgram quantities of protein using the principle of protein dye binding. Analytical Biochemistry 1976. 72, 248-54
2) Locke I C, Carpenter B G. Ion-dependency of the streptococcal deoxyribonuclease, “streptodornase,” an active constituent of the medicament Varidase®. Enzyme and Microbial Technology 2002. 31, 482-489.
3) Sinicropi D, Baker D L, Prince W S, Shifter K, Shak S. Colorimetric determination of DNase I activity with a DNA-methyl green substrate. Analytical Biochemistry 1994, 222, 351-358
4) Bexfield A, Nigam Y. Thomas S. Ratcliffe N A. Detection and partial characterisation of two antibacterial factors from the excretions/secretions of the medicinal maggot Lucilia sericata and their activity against methicillin-resistant Staphylococcus aureus. 2004, Microbes and infection
5) Mumcuoglu K Y. Miller J. Mumcuoglu M, Friger M, Tarshis M. Destruction of bacteria in the digestive tract of the maggot of Lucilia sericata. 2001. Journal of medical entomology, 38, 161-166
6) Hacker G. Redecke V, Hacker H. Activation of the immune system by bacterial CpG DNA. 2002, Immunology, 105, 245-251.
7) Hemmi H, Takeuchi O, Kawai T, Kaisho T, Sato S, Sanjo H, Matsumoto M, Hoshino K, Wagner H, Takeda K, Akita S. A Toll-like receptor recognises bacterial DNA. 2000, Nature, 408, 740-745.
8) Abreu M, Arditi M. Inate immunity and Toll-like receptors: clinical implications of basic science research. 2004, The Journal of Pediatrics, 421-429.
9) Sparwasser T, Koch E, Vabulas R M, Heeg K, Lipford G B, Ellwart J W, Wagner H. Bacterial DNA and immunostimulatory CpG oligonucleotides trigger maturation and activation of murine dendritic cells. 1998, European Journal of Immunology, 28, 2045-2054.
10) Sherman R A, Hall M J R, Thomas S. Medicinal maggots: An ancient remedy for some contemporary afflictions. 2000, Annual review of entomology, 45, 55-81.
11) Mumcuoglu K Y, Ingber A, Gilead L, Stessman J Friedmann R, Schulman H, Bichucher H, Ioffe-Uspensky, Miller J, Galun R et al. Maggot therapy for the treatment of intractable wounds. 1999, Pharmacology and Therapeutics, 623-627
12) Beasley W D, Hirst G, Making a meal of MRSA—the role of biosurgery in hospital acquired infection. 2004, Journal of hospital infection, 56, 6-9.
13) Chambers L, Woodrow S, Brown A P, Harris P D, Phillips D, Hall M, Church J C T, Pritchard D I. Degradation of extracellular matrix components by defined proteinases from the greenbottle larva Lucilia sericata used for the clinical debridement of non-healing wounds. British Journal of Dermatology, 2003, 148, 14-23.
14) Bexfield A, Nigam Y, Thomas S, Ratcliffe N A. Detection and partial characterisation of two antibacterial factors from the excretions/secretions of the medicinal maggot Lucilia sericata and their activity against methicillin-resistant Staphylococcus aureus. 2004, Microbes and infection
15) Horobin A J, Shakesheff K M, Woodrow S, Robinson C, Pritchard D I. Maggots and wound healing: an investigation of the effects of secretions from Lucilia sericata larvae upon interactions between human dermal fibroblasts and extracellular matrix components. 2003, British Journal f Dermatology, 148, 923-933.
16) Pullen R, Popp R, Volkers P, Fusgen I. Prospective randomized double-blind study of the wound debriding effects of collagenase and fibrinolysin/deoxyribonuclease in pressure ulcers. Age and Ageing, 2002, 31, 126-130.
17) Church J C (2001) Larval intervention in the chronic wound. EWMA 1 (2):10-13.
18) Horobin A J, Shakesheff K M, Pritchard D I (2005) Maggots and wound healing: an investigation of the effects of secretions from Lucilia sericata larvae upon the migration of human dermal fibroblasts over a fibronectin-coated surface. Wound Repair and Regeneration 13 (4): 422-433.
19) Locke, I. C., Cox, S. F. and Carpenter, B. G. 1997. Purification of a Streptococcal deoxyribonuclease by affinity chromatography based on a DNA-cellulose matrix.” Journal of Chromatography A 788: 75-80.
20) Whiting, R. F., Wei, L. and Stich, H. F. 1981. Chromosome-damaging activity of ferritin and its relation to chelation and reduction of iron. Cancer Research. 41(5):1628-36.
21) Toyokuni, S. and Sagripanti, J. L. 1992. Iron-mediated DNA damage: sensitive detection of DNA strand breakage catalyzed by iron. Journal of Inorganic Biochemistry. 47(3-4): 241-8.
22) Altschul S. F., Madden T. L., Schäffer A. A., Zhang J., Zhang Z., Miller W. and Lipman D. J. 1997. Gapped BLAST and PSI-BLAST: a new generation of protein database search programs. Nucleic Acids Research. 25:3389-3402.

1. A polypeptide isolated from insect larvae, or a synthetic analogue thereof, having the ability to degrade, denature, cut or cleave a nucleic acid.

2. The polypeptide of claim 1, wherein the larvae are larvae from Lucilia sericata.

3. The polypeptide of claim 1, wherein the polypeptide is isolated from the excretions or secretions of the insect larvae.

4. The polypeptide of claim 1, wherein the nucleic acid is DNA.

5. The polypeptide of claim 1, wherein the polypeptide degrades both prokaryotic and eukaryotic nucleic acids.

6. The polypeptide of claim 1, wherein the polypeptide degrades both bacterial and mammalian nucleic acids.

7.-10. (canceled)

11. The polypeptide of claim 1, wherein the polypeptide is a nuclease.

12. (canceled)

13. A method for the treatment or prophylaxis of infection or for the treatment of wounds in a human or animal, comprising;
administering to the human or animal the polypeptide of claim 1.

14. The polypeptide of claim 1, wherein the polypeptide is incorporated in a pharmaceutical composition comprising the polypeptide and a pharmaceutically acceptable carrier.

15. The polypeptide of claim 1, wherein the polypeptide is incorporated in a dressing.

16. A method for treating an infection in a human or animal comprising,
administering the polypeptide of claim 1 to the human or animal to adjuvantise a conventional antibiotic in the treatment of the infection.

17. (canceled)
